(19) United States
(12) Patent Application Publication
(10) Pub. No.: US 2026/0235618 A1
(43) Pub. Date: Aug. 13, 2026

(54) MICROBEADS WITH RATIOMETRIC LANTHANIDE ENCODING FOR DRUG SCREENING

(71) Applicants: CZ Biohub SF, LLC, San Francisco, CA (US); The Board of Trustees of the Leland Stanford Junior University, Stanford, CA (US); University of Copenhagen, Copenhagen (DK)

(72) Inventors: Polly M. Fordyce, Menlo Park, CA (US); Jamin B. Hein, Palo Alto, CA (US)

(21) Appl. No.: 19/138,382

(22) PCT Filed: Dec. 15, 2023
(86) PCT No.: PCT/US2023/084250

§ 371 Date: Jun. 12, 2025

Related U.S. Application Data
(60) Provisional application No. 63/387,748, filed on Dec. 16, 2022.

(51) Int. Cl. G01N 33/68; G01N 21/64; G01N 33/543; G01N 33/58

(52) U.S. Cl. CPC G01N 33/6845; G01N 21/6428; G01N 33/54313; G01N 33/582; G01N 2021/6439

(57) ABSTRACT

The disclosure provides methods and compositions for identifying agents that modulate binding of a protein to a peptide binding partner using spectrally encoded microbeads comprising binding site peptides in which there is a 1:1 linkage between sequences of the binding site peptide and the embedded spectral code.

### CROSS-REFERENCE TO RELATED APPLICATIONS

This application claims priority benefit of U.S. Provisional Application No. 63/387,748, filed Dec. 16, 2022, which is incorporated by reference for all purposes.

### STATEMENT AS TO RIGHTS TO INVENTIONS MADE UNDER FEDERALLY SPONSORED RESEARCH AND DEVELOPMENT

This invention was made with Government support under contract GM123641 awarded by the National Institutes of Health. The Government has certain rights in the invention.

### BACKGROUND

Libraries of spectrally encoded hydrogel beads with >1,000 unique codes (Microspheres with Ratiometric Barcode Lanthanide Encoding (MRBLEs; see, e.g., Gerver et al., Lab on a Chip 2012, Nguyen et al., Adv. Opt. Mat. 2017; and U.S. Pat. No. 10,240,145) and MRBLE production and functionalization processes (see, e.g., Feng et al., eng et al., Microsyst Nanoeng 6:109, 2020; and WO2002/094219) have been described. It has further been shown that peptides can be synthesized directly on the microbeads with a 1:1 linkage between the peptide sequence and the embedded spectral code. Such bead-bound peptide libraries can be pooled and incubated with fluorescently-labeled proteins to quantify protein/peptide interaction affinities (see, e.g., Nguyen et al., eLife 2019; 8:e40499).

### SUMMARY

It is understood that this summary features various aspects of the disclosure and is not provided as a comprehensive summary of all embodiments encompassed by the disclosure.

In one aspect, the disclosure provides methods and assay compositions that provide the ability to perform high-throughput analysis to identify agents, such as small molecules, that disrupt specific protein-peptide interactions. For example, in some embodiments, a protein that interacts with short linear binding site sequences within different protein binding partners can regulate different biological processes. In some instances, it can be desirable to selectively modulate activity of one binding partner of a protein of interest, but not another binding partner. For example, a signaling protein that interacts with binding site sequences within different protein binding partners can regulate different biological processes, such as apoptosis, cell cycle arrest, and cell cycle progression, through specific interactions with the different binding partners. In cancer therapy, or other therapeutic applications, it can be desirable to identify a therapeutic agent, such as a small molecule, that would disrupt only the protein-protein interactions of the signaling protein with a binding partner that drives cell cycle progression and/or cellular proliferation while maintaining the biological function mediated through other binding partners of the protein.

Accordingly, in one aspect, the disclosure provides a method of screening for an agent that modulates protein-protein interactions, wherein the method comprises providing a library comprising bead-bound peptides comprising binding site sequences for each different binding partner of a protein of interest, wherein a 1:1 known relationship exists between an embedded spectral code and the binding site sequence of the peptide attached to the bead. In some embodiments, there is a 1:1 relationship between embedded spectral code and the peptide sequence. In some embodiments, the library of bead-bound peptides is deposited into each well of an array comprising individual compartments. The populations of microbeads in the individual compartments can then be contacted with the protein of interest and an agent, e.g., a small molecule that modulates binding, or is a candidate for modulating binding, of the protein of interest to one or more binding partners of the protein. In some embodiments, following incubation of the protein of interest with peptide-bound microbeads in the presence of the candidate agent, beads from each well are iteratively imaged to detect the spectral code and the amount of signal generated from a detectable label, e.g., fluorescent label, directly or indirectly linked to the protein of interest, to quantify protein binding to each peptide in the presence of the candidate agent.

In some aspects, the disclosure features a method of identifying an agent that modulates interaction of a protein of interest with a binding site peptide, the method comprising providing a population of microbead-bound peptides comprising microbeads having distinguishable spectral codes, wherein a first microbead having a specific spectral code comprises a peptide having a first sequence that binds to the protein of interest, and a microbead having a different spectral code comprises a peptide having a second sequence that binds to the protein of interest, wherein the second sequence differs from the first sequence; contacting the population of microbeads with the protein of interest and a candidate agent for modulating binding of the protein interest to peptides present on the microbeads; detecting binding of the protein of interest to peptides on individual beads; and determining the spectral signature of individual microbeads, thereby determining the identity of the peptide linked to each bead. In some embodiments, the distinguishable spectral codes are lanthanide spectral codes. In some embodiments, the protein of interest comprises a detectable label. In some embodiments, the step of detecting binding of the protein of interest comprises contacting the population of microbeads with a detection reagent that binds to the protein of interest, wherein the detection reagent comprises a detectable label. In some embodiments, the contacting step comprises contacting a pooled population of microbeads with the protein of interest. In some embodiments, the method further comprises distributing the pooled population into an array configuration. In some embodiments, the lanthanide spectral codes are detecting by deep UV imaging. In some embodiments, the detectable label comprises a fluorescent label. In some embodiments, binding of the protein of interest to peptides present on individual microbeads is detected by image analysis. In some embodiments, the peptides are synthesized on the microbeads. In some embodiments, the detecting step comprises quantifying signal generated from the detectable label for protein bound to peptides on individual microbeads. In some embodiments, the method further comprises selecting an agent that decreases the amount of signal generated. In some embodiments, the agent is a small molecule. In other embodiments, the agent is a peptide, antibody, or aptamer. In some embodiments, the population of microbeads is a portion of a library of microbeads and the method further comprises detecting binding of the protein of interest to multiple populations of microbeads, wherein each population is from the same library and each population is present in an individual compartment, and wherein different candidate agents are added to the individual compartments.

In a further aspect, the disclosure provide an array comprising populations of lanthanide-encoded microbeads, each from the same library of microbeads, distributed into individual compartments, wherein each population comprises microbeads having distinguishable lanthanide spectral codes, wherein each microbead comprises a peptide comprising a binding site to a protein of interest attached thereto and wherein each microbead having the same spectral code comprises a peptide comprising the same binding site sequence and microbeads having different spectral codes comprise peptides comprising sites that differ in sequence. In some embodiments, each compartment further comprises the protein of interest and a candidate agent that modulates binding. In some embodiments, the candidate agent is a small molecule.

### BRIEF DESCRIPTION OF THE DRAWINGS

FIG. 1A-C. A) Illustration of an embodiment to discover a small molecule that disrupts a specific protein-protein interaction. The assay has the capability to distinguish which interaction site is targeted by a specific small molecule. B) Preparation of encoded beads and on-bead solid-phase peptide synthesis to synthesize peptides on beads with a 1:1 linkage between the peptide sequence and the embedded spectral cod. C) Assay overview: Encoded beads display different peptides binding to different binding sites on the protein of interest. The small molecule prevents binding of the protein to some peptide sequences. Deep UV imaging is used to image the beads in the lanthanide channels and identifying the embedded spectral code, thereby identifying the bead-displayed peptide sequence. Epi-fluorescence imaging detects the amount of protein binding to the encoded beads and the loss of binding to certain sequences in the presence of a small molecule.

FIG. 2 provides data illustrating the effect of the small molecule Apcin on the binding between Cdc20 and different peptide sequences. Binding was quantified by measuring the per-bead fluorescence intensity of a fluorescently-labeled antibody that recognizes bound Cdc20. The data demonstrated that Apcin blocks the binding site between Cdc20 and D-Box-containing sequences but not ABB-containing sequences. The control is the left bar for each of the peptides.

FIG. 3A-C illustrates: A) Simultaneous synthesis of bead bound peptides and soluble peptides for biological assays and quality control. B) Coupling of Fmoc-Glycine (an acid-resistant linker) to encoded beads and rink amide F-moc Glycine (an acid-labile linker) to magnetic beads prior to peptide synthesis. C) Peptide synthesis on encoded and magnetic beads. Deprotection of protected side chains using trifluoroacetic acid at the final step leads to cleavage of the rink amide linker and elution of peptides from magnetic beads that can be used for quality control. The acid-resistant Fmoc-glycine linker is unaffected such that peptides remain attached to spectrally encoded beads and these beads can be pooled, separated from magnetic beads and used for downstream multiplexed binding assays.

### DETAILED DESCRIPTION

### Terminology

The term “lanthanide” refers to elements 57-71 of the periodic table, namely lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), and lutetium (Lu). Reference to “lanthanide” can also include combinations of lanthanide elements, compounds containing lanthanide elements or their combinations, or ions containing lanthanide elements or their combinations.

The term “lanthanide nanoparticle” refers to a nanoparticle that includes a lanthanide and a host lattice. Lanthanide nanoparticles are sometimes referred to as “lanthanide nanophosphors.”

The term “host lattice” refers to a material that can accommodate the incorporation of lanthanide atoms or ions. When the host lattice is “lanthanide-doped,” it means that the host lattice material contains one or more lanthanides. For example, lanthanide dopants may be incorporated into a host lattice to provide lanthanide-doped yttrium orthovanadate (YVO4), lanthanide-doped oxides (for example, doped ZrO2, doped TiO2, doped BaTiO3), lanthanide-doped halides (for example, doped LaF3), lanthanide-doped phosphates (for example, dope LaPO4, doped LuPO4, or doped YbPO4), and lanthanide-doped strontium borates (for example, SrB4O7, SrB6O10, and Sr4B14O25), among others.

“Lanthanide encoded” or “spectrally encoded” microbeads described in the present disclosure contain lanthanide nanoparticles (Lns) and possess a detectable spectral signature, which is a combination of luminescent signals in the range of 350-850 nm emitted from lanthanide nanoparticles contained in a single microbead upon excitation with an appropriate wavelength of light, for example, UV light (such as 292 nm for excitation of downconverting lanthanides) or IR light (such as 980 nm for excitation of upconverting lanthanides). The luminescence intensity at a characteristic wavelength or wavelengths (for example, 620 nm, 630 nm, or 650 nm) for a particular lanthanide (for example, Eu) indicates the presence and quantity of the particular lanthanide in the source (for example, a microbead) from which the spectral signature originates. A “lanthanide encoded” or “spectrally encoded” microbead may include one or more different types of lanthanide nanoparticles, for example, 2 or more, 3 or more, 4 or more, 5 or more, 6 or more, 7 or more, 8 or more, 9 or more, or 10 or more, wherein each lanthanide nanoparticle has a different luminescence emission spectrum upon excitation. Signals from the combined luminescence spectra make up the spectral signature of a particular microbead and are mapped to a unique spectral signature code (or “spectral code”) during code deconvolution. Lanthanide nanoparticle spectra are typically characterized by narrow emission bands (also referred to as “signals”) in the visible region, making one species of material easily distinguishable from another. A lanthanide spectral signature of a microbead can therefore be designed based on the particular identity and relative amounts of lanthanides in the microbead. Lanthanide spectral signatures of microbeads described in the present disclosure can include one or more of an Eu signal, a Dy signal, a Sm signal, a Ce signal, a Tb signal, a La signal, a Pr signal, an Nd signal, a Gd signal, an Ho signal, an Er signal, a Tm signal, a Yb signal, a Pm signal, and a Lu signal.

As used herein, the term “microbead” or “microsphere” refers to a particle having one or more dimensions (e.g., length, width, diameter, or circumference) of about 1000 μm or less, e.g., less than about 500 μm, 100 μm, or 10 μm. Microbeads may have a generally spherical shape or a non-spherical shape. Microbeads used in the methods of the present disclosure are characterized by a detectable spectral signature as described in more detail below. A “plurality” of microbeads refers to a population of microbeads ranging in size from a few microbeads to thousands of microbeads, or more. The terms “microsphere” and “microbead” are used interchangeably in the present disclosure regardless of whether the bead has a generally spherical or non-spherical shape.

The term “amino acid” encompasses naturally occurring amino acids as well as non-naturally occurring amino acids, including amino acid analogs and derivatives. Amino acids include naturally occurring proteogenic L-amino acids; D-amino acids; chemically modified amino acids such as amino acid analogs and derivatives; naturally occurring nonproteinogenic amino acids such as norleucine, p-alanine, or ornithine; and chemically synthesized compounds having amino acid characteristics. Amino acids may be referred to herein by either their commonly known three letter symbols or by the one-letter symbols recommended by the IUPAC-IUB Biochemical Nomenclature Commission.

The term “peptide” is used herein to refer to a polymer of amino acid residues. The term applies to amino acid polymers in which one or more amino acid residue is an artificial chemical mimetic of a corresponding naturally occurring amino acid, as well as to naturally occurring amino acid polymers and non-naturally occurring amino acid polymers. Thus, a peptide used in a panel of peptides to analyze binding of a protein of interest to binding partner peptides as described herein can comprise naturally occurring and/or synthetic amino acids, including analogs and amino acid mimetics that function in a manner similar to the naturally occurring amino acids.

The term “label” or “detectable label”, as used herein, refers to any atom or molecule that can be used to provide a detectable and/or quantifiable signal. In some embodiments, the label can be attached, directly or indirectly, to a biomolecule. Labels include, but are not limited to, fluorophores, chromophores, radioisotopes, mass labels, electron dense particles, magnetic particles, spin labels, molecules that emit chemiluminescence, electrochemically active molecules, enzymes, cofactors, oligonucleotides, and enzyme substrates.

As used herein, “detection” of a signal includes embodiments in which a signal is quantified.

An “array configuration” as used herein refers to a collection of single compartments. An array configuration may be an “ordered array” in which the compartments are addressable and can be assigned to known locations.

A “compartment” as used herein in the context of distributing microbeads refers to any partially or fully enclosed space that separates pools of microbeads or separates individual microbeads. Thus, a compartment can include microwells, microfluidic chambers, and the like.

### Introduction

The present disclosure provides methods and compositions that allows high-throughput screening of the ability of a compound to modulate binding of a protein of interest to peptides comprising different binding sites, e.g., different binding sites from naturally occurring binding partners of the protein of interest, for the protein. The methods employ libraries of peptides bound to spectrally encoded microbeads in which there is a 1:1 correspondence of the binding site sequence present in a peptide and the spectral code of the microbead. This allows for efficient interrogation of the effects of a candidate agent on binding of the protein of interest to a plurality of peptides bound to the microbeads.

A protein of interest can be any protein that has a binding partner, but typically is a protein that in nature has more than one binding partner to which the protein can bind. The binding partners have binding sites for the protein of interest that generally share a sequence motif or biophysical properties that confer specificity for the interaction. For example, in some embodiments, the protein of interest participates in signal transduction. In some embodiments, a protein of interest participates in protein degradation pathways or other regulatory pathway. In some embodiments, a protein of interest binds to naturally occurring variants of a protein, e.g., allelic variants, or related protein encoded by different members of a family of genes.

### Binding Site Peptide

In the context of this disclosure, a “binding site peptide” refers to a peptide that comprises a binding site sequence, or variant of a binding site sequence, from a binding partner of a protein of interest. A “binding site peptide” is also referred to herein as a “peptide binding partner”.

A “binding partner” refers to a molecule that comprises a protein component that binds to a protein of interest via a specific binding site sequence present in the binding partner. In the present disclosure, a binding site typically comprises a linear sequence. A protein of interest may have different binding partners, i.e., may bind to different binding partner proteins that comprise binding sites that differ. Binding site sequences, however, generally comprise at least some shared amino acids residues of a binding motif recognized by the protein of interest or some shared structural or biophysical features that confer specificity.

In some embodiments, a binding site peptide may comprise selected or random mutations in the binding site sequence. In some embodiments, a series of mutations is introduced into a peptide to further evaluate interactions with the protein of interest in the presence of an agent that modulates binding, or is a candidate agent for modulating binding, of the protein of interest to the binding site peptide.

Binding site peptides can vary in length, e.g., from 7 to 25 amino acids in length, or from 9 to 21 amino acids in length. In some embodiments, a peptide may be up to 50 amino acids in length, or up to 100 or 150 amino acids in length, and at least 5 amino acids in length. In some embodiments, a binding site peptide is 9 to 50 amino acids in length. In some embodiments, a binding site peptide can comprise additional amino acids at the C-terminal and/or N-terminal ends.

### Agents That Modulate Binding

Any agent (also referred to as a modulator) that can influence binding of a protein of interest to a binding site peptide may be evaluated using peptide-linked spectrally encoded microbeads as described herein. In some embodiments, potential modulators are screened for the ability to disrupt binding of the protein of interest to binding site peptides. Alternatively, in some embodiments, potential modulators are screened for the ability to enhance binding of a protein of interest to one or more binding site peptides.

In some embodiments, the modulator or candidate agent is a “small molecule”. These include, but are not limited to, organic or inorganic compounds that typically have a molecular weight of less than about 5,000 Da. In some embodiments, the small molecule is a small organic compound that has a molecular weight of less than 1,000 Da. In some embodiments, the agent is a small molecule having a molecular weight of about 100 to about 1,000 Da or about 500 to about 5,000 Da. As used here, “about” refers to a range within 10% or within 20% of the indicated value.

Other modulators or candidate agents that may modulate binding include peptide agents. In some embodiments, a peptide modulator is a relatively small peptide, e.g., from 5-100 amino acids in length.

In some embodiments, a candidate agent or modulator is an antibody. The term “antibody” encompasses full-length antibody formats, e.g., IgG, and functional fragments of antibodies that retain antigen binding specificity, including multimeric and monomeric forms. The term encompasses polyclonal and monoclonal antibody preparations, and engineered antibodies. “Antibody” thus also refers to binding formats including diabodies, triabodies, tetrameric forms, single domain antibodies and the like. A functional fragment can be a portion of an antibody such as a F(ab′)₂, Fab′, Fab, Fv, or can be an engineered binding fragments, such as an scFV.

In some embodiments, a candidate agent or modulator is a peptide aptamer. Aptamers interact with their targets by recognizing a specific three-dimensional structure. Peptide aptamers are composed of a short variable peptide loop attached at both ends to a protein scaffold such as the bacterial protein thioredoxin-A. A peptide aptamer specific to a target of interest may be selected using any method known by the skilled person such as the yeast two-hybrid system or phage display. Peptide aptamers may be produced by chemical synthesis or recombinantly produced.

In some embodiments, a candidate agent or modulator is a nucleic acid aptamer. Nucleic acid aptamers are a class of small nucleic acid ligands that are composed of RNA or single-stranded DNA oligonucleotides folded into a three-dimensional structure that have high specificity and affinity for their targets. For example, Systematic Evolution of Ligands by Exponential enrichment (SELEX) technology can be used to obtain aptamers specific to a particular molecular target. Nucleic acid aptamers can be produced by as chemical synthesis or in vitro transcription for RNA aptamers. Nucleic acid aptamers include DNA aptamers, RNA aptamers, XNA aptamers (nucleic acid aptamer comprising xeno nucleotides) and L-RNA aptamers.

### Synthesis of Peptides on Microbeads

Synthesis of peptide directly on microbeads is known. See, for example, the methods detailed in WO2021252735 and WO2022094219, each incorporated by reference. In brief, such methods allow for inclusion of reactive (or functional) groups on a surface of the polymeric microspheres during microsphere solidification, i.e., the lanthanide-encoded polymeric microbeads can be functionalized as a part of the production process without adding additional steps after microbeads are performed. Functionalization of lanthanide-encoded polymeric microbeads can be accomplished by including a suitable amphipathic compound in a second, immiscible fluid used for droplet generation. Such a suitable amphipathic compound is capable of covalently bonding with the microbead matrix component during the solidification step. A suitable amphipathic compound includes one or more reactive groups that remain free after covalent binding of the amphipathic compound to the surfaces of the polymeric microbeads, and these free reactive groups can be used for subsequent attachment of molecules or moieties of interest to the microbeads, e.g., a binding moiety. Molecules of the suitable amphipathic compound included in the continuous phase are driven to and remain at the interface of the immiscible fluid and the matrix component-and lanthanide particle-containing fluid after droplet formation, with the hydrophobic parts of the amphipathic molecules facing a hydrophobic fluid (which may be the matrix and nanoparticle-containing fluid or the immiscible fluid), and the hydrophilic parts of the amphipathic molecules facing a hydrophilic fluid (which may be the matrix and nanoparticle-containing fluid or the immiscible fluid). In some embodiments, polymeric microbeads can be functionalized by two or more different reactive groups during their production by using, for example, an amphipathic compound with two or more reactive groups. In another example, multiple amphipathic compounds with different reactive croups can be added to the continuous phase during droplet generation.

In other embodiments, microbeads having various functional groups that can be used for on-bead synthesis or chemical coupling of peptide are generated using a technique described by Feng et al., Microsyst Nanoeng 6: 109, 2020, which is incorporated by reference. This method employs mixing of lanthanide-polymer mixture followed by droplet generation using a single-layer, parallel flow-focusing device, with polymerization of droplet in batch off of the chip. Copolymers bearing functional groups typically used for bioconjugation are localized to the surface of the hydrogel matrix during droplet generation and these polymers are covalently cross-linked in place during bead polymerization.

Some non-limiting examples of compounds that can be used for microbead functionalization and included in the second fluid are: for functionalization with carboxyl groups, unsaturated fatty acids, such as 10-undecenoic acid, 4-pentenoic acid, 5-hexenoic acid, 6-heptenoic acid, 7-octenoic acid, 8-nonenoic acid, or 9-decenoic acid; for functionalization with amino groups, amphiphathic amines, such as pent-4-enylamine, N-(3-Aminopropyl)methacrylamide, 2-Aminoethyl methacrylate, or N-(2-aminoethyl) methacrylamide; for functionalization with azide groups, 3-azidopropyl acrylate or 3-azidopropyl methacrylate; for functionalization with hydroxyl groups, 4-Penten-1-ol; for functionalization with hydrazide groups, hydrazido acrylate; for functionalization with chloromethyl groups, chloromethyl acrylate.

In some embodiments, a reactive group comprises an amino group, and the method further comprises performing solid-phase peptide synthesis on a surface of the polymeric microbeads. Thus, for example, in some embodiments, beads having a specific spectral code signature are collected into a single compartment, e.g., a well of a microwell plate, and the plate is transferred to a high-throughput solid phase peptide synthesizer to synthesize peptides on beads with a 1:1 linkage between the bead spectral and substrate peptide. The direct incorporation of the reactive group, (e.g., pent-4-eylamine) during bead production ensures that the bead-peptide linkage is covalently coupled to the rest of the matrix and remains stable during peptide synthesis and side chain deprotection.

In some embodiments, magnetic polymeric beads with the same chemical composition as the spectrally encoded beads, but having an acid-labile linker, are incorporated into the bead pool used for solid phase synthesis The beads can then be separated from the rest of the pool using a magnet and the material eluted for downstream characterization, e.g., by mass spectrometry and quality control.

In some embodiments, binding site peptides comprising phosphorylated residues are synthesized directly on microbeads for evaluation of binding activity.

In some methods for producing derivatized microbeads, a reactive group comprises a hydroxyl group, and the method further comprises performing cyanogen bromide-activated covalent coupling of an amino acid, a peptide or a protein to the reactive group. In some exemplary methods for producing derivatized microbeads, a reactive group comprises a chloromethyl group, and the method further comprises performing covalent coupling of an amino acid, a peptide, a protein or other amino-group containing molecule to the reactive group. In some exemplary methods for producing derivatized microbeads, a reactive group comprises an azide group, and the method further comprises performing covalent coupling of a molecule or moiety of interest via click chemistry to the reactive group.

In other embodiments, biotin-modified peptides may be loaded onto the microbeads via interaction with streptavidin coated on the surface of the spectrally encoded microbeads.

In some embodiments, beads are distributed to one or more compartments for binding activity assessment. In some embodiments, peptide-linked microbeads are pooled for binding activity assay.

### Spectral Signature

In general, each of the spectral signatures employed in an assay contains signals generated from predetermined amounts of two or more lanthanides (e.g., two or more Eu-, Dy-, Sm-, Ce-, Tb-, La-, Pr-, Nd-, Gd-, Ho-, Er-, Tm-, Yb-containing materials such as nanoparticles). Lanthanide materials in the microbeads can be excited with UV light (e.g., 275 nm or 292 nm) and emitted luminescent signals can be detected in the range of 400-800 nm (e.g., 435 nm, 474 nm, 527 nm, 536 nm, 546 nm, 572 nm, 620 nm, 630 nm, 650 nm, or 780 nm). A signal in a spectral signature may be measured as an absolute value or as a ratio of the signal to another reference signal. As a non-limiting example, a set of unique spectral signatures can be prepared with microparticles that contain europium-dope yttrium orthovanadate (YVO₄:Eu) to generate a reference signal and varying amounts of YVO₄:Dy, YVO₄:Sm, YVO₄:Tm, and LaPO₄:CeTb.

In some embodiments, binding site peptide-linked beads are distributed in an array configuration and a spectral signature of a microbead can be determined by imaging, e.g., deep UV imaging for lanthanides.

In some embodiments, binding activity is assessed using pools of peptide-bound microbeads and the spectral signature is determined iteratively.

In some embodiments, lanthanides embedded in different host matrices excite at low energy light, e.g., 980 nm IR, and emit in visible light. In applications employing such “up-converting” lanthanide:host matrix combinations, the spectral signature is determined via infrared imaging.

### Assessment of Binding Activity

Binding activity of a protein of interest to a population of microbeads in the presence of a modulator or candidate agent is evaluated by detecting signal generated from a label directly or indirectly attached to the protein of interest.

In one illustrative assay, a library of peptide-bound microbeads is generated by synthesizing binding site peptides directly on microbeads. Beads are pooled and then incubated with the protein of interest in the presence or absence of a modulator or candidate biding agent. The beads are then typically washed and the amount of signal from a label directly or indirectly attached to the protein of interest that is bound to each bead is determined. In some embodiments, portions of the library comprising a plurality of library members are distributed to individual compartments to compare the ability of different candidate agents or modulators to influence binding of the protein of interest to the members of the library. Thus, for example, signal bound to individual beads in a compartment containing a portion of the library, the protein of interest, and a modulator of candidate of interest can be conveniently compared to signal bound to individual beads in one or more parallel compartments having members of the same binding peptide-microbead library, but in which different agents are tested for the ability to modulate binding of the protein of interest.

### Techniques to Further Illustrate Aspects of the Disclosure

In some embodiments, highly monodisperse beads comprised of a PEG-DA hydrogel matrix using microfluidic droplet generators are produced (e.g., Feng et al., Microsystems & Nanoengineering 2020). These beads can be spectrally encoded via the ratiometric incorporation of different lanthanide nanophosphors and functionalized with amino groups. In the present disclosure, lanthanides are advantageously used for spectral signatures. In some embodiments, e.g., using small libraries with a small number of binding site peptides, e.g., fewer than 200, other agents that generate a color signal, such as fluorescent molecules, may be embedded in microbeads for spectral encoding.

After production, beads containing a given code are collected in a single compartment, e.g., a well in a microwell plate, such as a 96-well plate, so that the entire plate can be transferred to a high-throughput solid-phase peptide synthesizer to synthesize peptides on beads with to provide the 1:1 linkage between the peptide sequence and the embedded spectral code. Direct incorporation of the chemical functional reagent for linking an amino group (e.g., pent-4-enylamine) during bead production ensures that the bead-peptide linkage is covalently coupled to the rest of the hydrogel matrix and remains stable during peptide synthesis and side-chain deprotection.

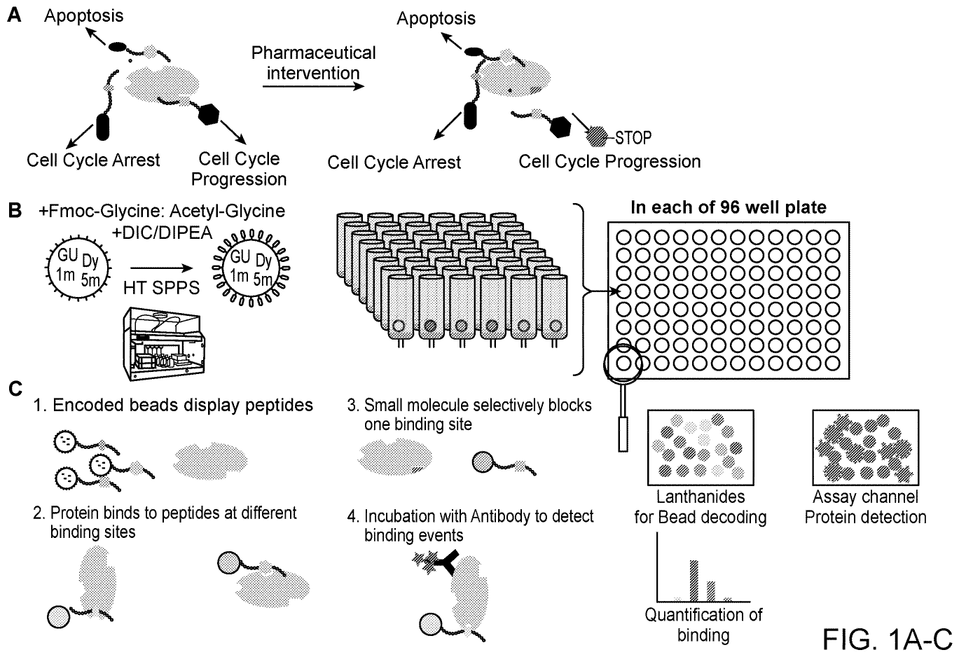

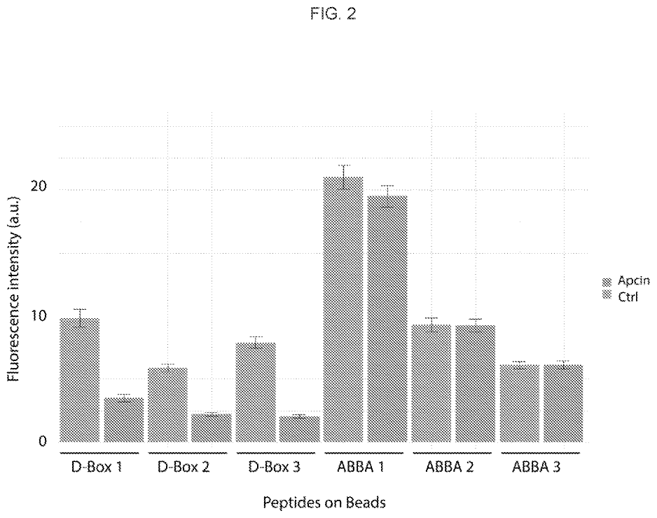

For illustration, binding site peptides can be from proteins that are binding partners of any protein of interest, e.g., a signaling protein. For example, a signaling protein may interact with short linear motifs within three different protein partners to regulate three different biological processes, such as apoptosis, cell cycle arrest, and cell cycle progression (illustrated in FIG. 1A). Thus, for example, in a screen for an anti-cancer therapy, candidate agents, e.g., small molecules, can be identified that have minimal off-target effects and disrupt only the protein/protein interaction that drives cell cycle progression and proliferation while leaving the other protein functions intact (FIG. 1A). In some embodiments, a bead-bound peptide library prepared as described herein can be generated in which peptides are specific for overlapping regions and variants within each different protein partner binding site with a 1:1 relationship between embedded spectral code and displayed peptide sequence (FIG. 1B). In some embodiments, such a bead, or pool of such beads, is deposited in a compartment, e.g., a microwell plate such as a 96-well plate, with a signaling protein and a candidate agent to be evaluated, such as a small molecule. In some embodiments, the signaling protein can be directly attached to a detectable label, e.g., a fluorescent label. In alternative embodiments, the protein can be indirectly labeled, e.g., using a labeled secondary antibody and an unlabeled primary antibody to the protein of interest. After incubating, beads from each well are iteratively imaged, e.g., via deep UV imaging for lanthanide channels and fluorescence channels for quantification of fluorescent label, to directly quantify how much protein binds each peptide in the presence (and absence for control purposes) of each agents, e.g., small molecule drug. The analysis thus shows the amount of protein bound to a given bead with a certain sequence and the level to which binding to specific binding site sequences is altered in the presence of a small molecule (see, e.g., FIG. 1C). Use of multiple peptide sequences (e.g., for different binding sites or different affinities) can serve as an internal control and provide the ability to quantitatively assess a loss of signal without concerns regarding assay quality (e.g., FIG. 2).

This assay is easily extendible to further quantifying cross-talk between potential signaling protein targets as well as evaluating how human allelic variants may alter efficacy. For example, for quantifying cross-talk, the influence of the small molecule, or a biological agent, on a protein closely related to a protein of interest can be evaluated in the same assay and compartment as the protein of interest by using a different fluorophore for detection of the desired protein vs the closely related off-target protein.

In some embodiments, to evaluate effects of human allelic variants on the ability of a protein comprising a binding site to interact with variant peptides targets, allelic variants of recognized peptide binding sites can be synthesized directly on beads as part of the microbead library. Although the absence of a signal is typically an unfavorable readout for biological assays, the ability to simultaneously detect the interaction between multiple peptides and the target proteins as described in the present disclosure allows for evaluating loss of binding through extensive internal assay controls. Thus, the present disclosure provides a screening method to target protein-protein interactions, e.g., for drug development, by providing robust multiplexing capabilities.

### Illustrative Binding Assay

The ubiquitin-proteasome pathway uses E3 ubiquitin ligases to attach the small protein ubiquitin to lysine residues on substrate proteins as a signal for proteasomal degradation. In order to recognize the correct protein, E3 ligases often bind proteins, commonly referred to as substrate adaptors, that recognize specific substrates. Cdc20 is a substrate adapter for the E3 ubiquitin ligase APC/C and important for cell cycle regulation by targeting proteins for degradation at the right time. Cdc20 has three different binding sites for short sequences to recognize substrate or regulatory proteins containing either D-Box (RxxLxxxxN), KEN-Box (KEN) or ABBA (Fx(I/L)Fx(D/E)) motifs. So far, there is only one small molecule, called Apcin, that targets the D-Box interaction site. In this example, we generated a peptide library as described herein covering peptides from different proteins with a DBox or an ABBA motif and demonstrated that addition of Apcin selectively prevented the binding of Cdc20 to D-Box-containing peptides, but not ABBA-motifs (FIG. 2).

### Example 2. Quality Control for On-Bead Peptide Synthesis

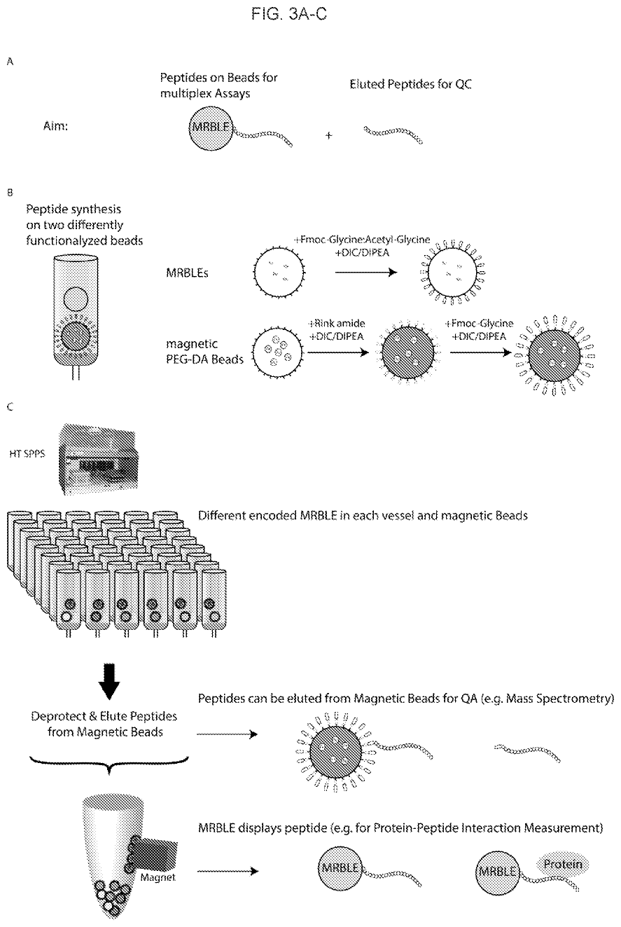

FIG. 3A-C illustrates a method for quality control for on-bead peptide synthesis. As described above, a microfluidic chip is used to produce beads by embedding lanthanide nanphosphors in a PEG-DA hydrogel matrix with pent-4-enylamine (as a chemical handle for peptide synthesis). Magnetic beads using Fe₃O₄ nanoparticles instead of lanthanides for embedding into a PEG-DA hydrogel functionalized with pent-4-enylamine can also be prepared. The magnetic beads can be isolated from the lanthanide-encoded beads using a magnet. Prior to peptide synthesis, an acid-labile linker (Rink amide) is attached to the magnetic beads so that peptides are cleaved from the bead matrix upon exposure to acid during the final global side chain deprotection step after peptide synthesis. In particular, amine functionalized magnetic beads can be treated with DIC/DIPEA to attach rink amide as an acid labile linker. An acid-resistant linker (Fmoc-Glycine) is then attached to both amine-functionalized encoded beads and the rink amide magnetic beads. The encoded and magnetic beads can be pooled and peptides synthesized on an automated solid-phase peptide synthesizer (FIG. 3C). After completion, the side protecting groups of the peptides can be removed using a mixture of TFA, TIPS and H2O. This treatment also results in the selective cleavage of the peptides attached to the magnetic beads. These peptides can be collected, washed and analyzed using LC/MS; the remaining spectrally encoded beads with peptides still attached can be collected and used for on-bead multiplexed binding assays. An example of LC/MS data providing quality control information about the synthesis of one peptide is presented in FIG. 3D.

It is understood that the examples and embodiments described herein are for illustrative purposes only and that various modifications or changes in light thereof will be suggested to persons skilled in the art and are to be included within the spirit and purview of this application and scope of the appended claims.

1. A method of identifying an agent that modulates interaction of a protein of interest with a binding site peptide, the method comprising providing a population of microbead-bound peptides comprising microbeads having distinguishable spectral codes, wherein a first microbead having a specific spectral code comprises a peptide having a first sequence that binds to the protein of interest, and a microbead having a different spectral code comprises a peptide having a second sequence that binds to the protein of interest, wherein the second sequence differs from the first sequence;
contacting the population of microbeads with the protein of interest and a candidate agent for modulating binding of the protein interest to peptides present on the microbeads;
detecting binding of the protein of interest to peptides on individual beads; and
determining the spectral signature of individual microbeads, thereby determining the identity of the peptide linked to each bead.

2. The method of claim 1, wherein the distinguishable spectral codes are lanthanide spectral codes.

3. The method of claim 1, wherein the protein of interest comprises a detectable label.

4. The method of claim 1, or wherein the step of detecting binding of the protein of interest comprises contacting the population of microbeads with a detection reagent that binds to the protein of interest, wherein the detection reagent comprises a detectable label.

5. The method of claim 1, wherein the contacting step comprises contacting a pooled population of microbeads with the protein of interest.

6. The method of claim 5, further comprising distributing the pooled population into an array configuration.

7. The method of claim 1, wherein the lanthanide spectral codes are detecting by deep UV imaging.

8. The method of claim 1, wherein the detectable label comprises a fluorescent label.

9. The method of claim 1, wherein binding of the protein of interest to peptides present on individual microbeads is detected by image analysis.

10. The method of claim 1, wherein the peptides are synthesized on the microbeads.

11. The method of claim 1, wherein the detecting step comprises quantifying signal generated from the detectable label for protein bound to peptides on individual microbeads.

12. The method of claim 1, further comprising selecting an agent that decreases the amount of signal generated.

13. The method of claim 1, wherein the agent is a small molecule.

14. The method of claim 1, wherein the agent is a peptide, antibody, or aptamer.

15. The method of claim 1, wherein the population of microbeads is a portion of a library of microbeads and the method further comprises detecting binding of the protein of interest to multiple populations of microbeads, wherein each population is from the same library and each population is present in an individual compartment, and wherein different candidate agents are added to the individual compartments.

16. An array comprising populations of lanthanide-encoded microbeads, each from the same library of microbeads, distributed into individual compartments, wherein each population comprises microbeads having distinguishable lanthanide spectral codes, wherein each microbead comprises a peptide comprising a binding site to a protein of interest attached thereto and wherein each microbead having the same spectral code comprises a peptide comprising the same binding site sequence and microbeads having different spectral codes comprise peptides comprising sites that differ in sequence.

17. An array of claim 16, wherein each compartment further comprises the protein of interest and a candidate agent that modulates binding.

18. The array of claim 16, wherein the candidate agent is a small molecule.
